(19) United States
(12) Patent Application Publication
(10) Pub. No.: US 2025/0261676 A1
(43) Pub. Date: Aug. 21, 2025

(54) FUNGAL BIOMASS AND METHOD OF PRODUCING THEREOF

(71) Applicant: ENIFERBIO OY, Espoo (FI)

(72) Inventors: Anssi RANTASALO, Espoo (FI); Simo ELLILÄ, Espoo (FI); Joosu KUIVANEN, Tampere (FI)

(73) Assignee: ENIFERBIO OY, Espoo (FI)

(21) Appl. No.: 18/859,218

(22) PCT Filed: May 11, 2023
(86) PCT No.: PCT/FI2023/050259

§ 371 Date: Oct. 23, 2024

(30) Foreign Application Priority Data
May 12, 2022 (FI) 20225418

(51) Int. Cl. A23L 31/00; C12N 1/14; C12N 15/01; C12R 1/79

(52) U.S. Cl. CPC A23L 31/00; C12N 1/145; C12N 15/01; C12R 2001/79

(57) ABSTRACT

A method of producing a fungal biomass includes culturing genetically changed genomes of filamentous fungus on a medium, which has non-metabolizable glucose analogue and a growth carbon source comprising glycerol, isolating improved fungal strains from the cultured genetically changed genomes, wherein the improved fungal strains exhibit growth on the medium, pre-cultivating the isolated improved fungal strains, fermenting the pre-cultivated improved fungal strains with a feedstock to obtain a fermentation broth, wherein the feedstock has a fermentation carbon source with glycerol, collecting the fermentation broth, and separating the fungal biomass from the fermentation broth.

### TECHNICAL FIELD

The present disclosure relates to a fungal biomass and methods of producing a fungal biomass.

### BACKGROUND

Over the past few years, the demand for new alternative protein sources has increased rapidly both for animal feed and human nutrition as a result of ethical and environmental concerns associated with conventional sources of proteins, including plants and animals. In addition to conventional sources of proteins, algae, insects and microbes are one of the most potential future sources of protein. Notably, the protein component derived from the whole microbial biomass is referred to as single cell protein (SCP). Another potential future source of SCP includes fungal organisms and the protein component derived therefrom is specifically referred to as mycoprotein. Notably, a well-known example of mycoproteins in commercial production is Quorn.

Typically, one of the factors limiting the large-scale use of SCPs is the cost of production thereof. One approach to lower the production costs is to use low-value side streams as feedstock in the production process. However, these side streams, such as vinasse or stillage from bioethanol production, consist of a mixture of different carbon sources (such as residual sugars, oligosaccharides (maltose) and glycerol) which may not be optimal for microbial growth. As a result, growth rate in a batch fermentation and biomass concentration in a continuous fermentation are not optimal.

Side streams from bioethanol production, vinasse or stillage (residues from ethanol distillation), are potential feedstocks for SCP production but consist of a mixture of different carbon sources such as residual sugars, oligosaccharides (maltose) and glycerol. By nature, microbial organisms including those cultivated for SCP production preferably first use most rabidly metabolizable carbon sources i.e. free sugars and only then begin to utilize other carbon sources such as glycerol and oligosaccharides. As a result, growth rate in a batch fermentation and biomass concentration in a continuous fermentation are not optimal. In case of continuous fermentation, especially at high dilution rate, some of the carbon sources remain unutilized and leak out from the fermentation process which us undesirable. Furthermore, SCP produced with the mentioned side streams cannot be used in food products.

The problem of suboptimal carbon utilization can be addressed by increasing a residence time of feedstock in the fermenter i.e. lowering the dilution rate in a continuous fermentation or by increasing cultivation time in a batch process. However, increment of residence time, may have adverse effects on the product quality in mycoprotein (i.e. decreased protein content). Moreover, in case of continuous fermentation, especially at high dilution rate, some of the carbon sources remain unutilized and leak out from the fermentation process which is undesirable. This results in lower productivities and yields than could theoretically be achieved, and further in higher production costs, especially when using the continuous fermentation method. Alternative methods for improving substrate utilization include feedstock dilution or recirculation. However, diluting the feedstock leads to significant water consumption and effluent volumes while recirculation of feedstock is technically and economically challenging e.g. due to high aseptic requirements.

Reportedly, a few fungal strains with improved ability to utilize glycerol in aerobic fungal (yeast or filamentous fungus) fermentation have been identified. Such strains may include, but not limit to, Saccharomyces cerevisiae, and other genetically engineered fungal strains. However, such strains are grown using a pure glycerol, not a feedstock with a mixture of carbon sources, as carbon source to produce biochemicals or to avoid glycerol formation by yeast itself in ethanol fermentation. However, the utilization rate of some substrates commonly present in significant amounts in feedstock, especially glycerol and maltose, remains low during fast dilution rates in a continuous fermentation. This leads to suboptimal feedstock utilization which is reflected in higher production cost and feedstock requirements, as well as causing elevated effluent biochemical oxygen demand (BOD) levels.

Moreover, some fungal strains with a disrupted carbon catabolite repression system (resulting from UV mutagenesis in fungi followed by selection for growth in the presence of 2-deoxy-d-glucose) have also been reported to create glucose-insensitive strains with improved enzyme secretion capabilities. However, such fungal strains have not been reported with improved glycerol or maltose utilization capacity, via disrupted carbon catabolic repression system, for the purpose of producing mycoprotein or capable of using side streams from bioethanol production (vinasse or stillage) as feedstock in for example continuous fermentation.

Therefore, in light of the foregoing discussion, there exists a need to overcome the aforementioned drawbacks associated with the conventional methods of producing a fungal biomass having a desirable yield and nutritional value.

### SUMMARY

The present disclosure seeks to provide a method of producing a fungal biomass. The present disclosure also seeks to provide use of a fungal biomass obtained by the aforementioned method as a food ingredient or in whole foods. An aim of the present disclosure is to provide a solution that overcomes at least partially the problems encountered in prior art. Advantageous features are set out in the appended dependent claims.

In a first aspect, an embodiment of the present disclosure provides a method of producing a fungal biomass comprising: culturing genetically changed genomes of filamentous fungus on a medium, which comprises non-metabolizable glucose analogue and a growth carbon source comprising glycerol, isolating improved fungal strains from the cultured genetically changed genomes, wherein the improved fungal strains exhibit growth on the medium, pre-cultivating the isolated improved fungal strains, fermenting the pre-cultivated improved fungal strains with a feedstock to obtain a fermentation broth, wherein the feedstock comprises a fermentation carbon source comprising glycerol, collecting the fermentation broth, and separating the fungal biomass from the fermentation broth.

In a second aspect, an embodiment of the present disclosure provides use of the fungal biomass obtained by the aforementioned method as a food ingredient or in whole foods.

Embodiments of the present disclosure substantially eliminate or at least partially address the aforementioned problems in the prior art, and enable an improved and efficient method for producing the fungal biomass. Moreover, the method enables an increased productivity and yield in the production of the fungal biomass as well as mycoproteins therefrom. Moreover, the method employs the bioreactor that enables the use of low-value side streams as the feedstock having a mixture of different fermentation carbon sources comprising both the preferred (for example glucose) and the non-preferred (for example glycerol and maltose) carbon sources.

Additional aspects, advantages, features and objects of the present disclosure would be made apparent from the drawings and the detailed description of the illustrative embodiments construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. Embodiments of the present disclosure will now be described, by way of example only, with reference to the following diagrams wherein:

FIG. 1 is a flowchart of steps of a method of producing a fungal biomass, in accordance with an embodiment of the invention;

FIG. 2 is a schematic illustration of steps of a method of producing a fungal biomass, in accordance with an embodiment of the invention; and

FIG. 3A is an illustration of steps of a method of cultivating the isolated fungal strains with a disrupted carbon catabolite repression for producing a fungal biomass via a batch bioreactor and via a continuous bioreactor, in accordance with an embodiment of the present disclosure.

FIG. 3B is an illustration of steps of a method of cultivating the isolated fungal strains with a disrupted carbon catabolite repression for producing a fungal biomass via a batch bioreactor, in accordance with an embodiment of the present disclosure.

### DETAILED DESCRIPTION OF EMBODIMENTS

The following detailed description illustrates embodiments of the present disclosure and ways in which they can be implemented.

In a first aspect, an embodiment of the present disclosure provides a method of producing a fungal biomass comprising: culturing genetically changed genomes of filamentous fungus on a medium, which comprises non-metabolizable glucose analogue and a growth carbon source comprising glycerol, isolating improved fungal strains from the cultured fungal strains, wherein the improved fungal strains exhibit growth on the medium, pre-cultivating the isolated improved fungal strains, fermenting the pre-cultivated improved fungal strains with a feedstock to obtain a fermentation broth, wherein the feedstock comprises a fermentation carbon source comprising glycerol, collecting the fermentation broth, and separating the fungal biomass from the fermentation broth.

The present disclosure provides the aforementioned method of producing the fungal biomass. The method employs the genetically changed genomes of filamentous fungus having improved capacity to use a mixture of carbon sources supplied via low-value side streams, for example side streams from thin stillage or vinasse from bioethanol production, as feedstock. In this regard, the method employs disrupting the carbon catabolite repression in such filamentous fungus and pre-cultivating the isolated improved fungal strains for increasing a growth rate thereof during a fermentation process. Moreover, the method may employ a variety of fermentation processes, such as batch or continuous fermentation, for providing an efficient fermentation of the isolated fungal strain and separating the fungal biomass based on an application thereof. The present disclosure is related to increasing growth rate for example in a batch fermentation and biomass concentration for example in a continuous fermentation and thereby improving productivity and yield in SCP production. In particular, the present disclosure is related to how to improve the utilization of non-metabolizable carbon sources, for example glycerol and maltose as fermentation carbon sources, in an aerobic fungal batch and continuous fermentations. According to test results, using a growth carbon source comprising glycerol during culturing fungal strains according to the present disclosure results in improved glycerol utilization capacity during fermentation for obtaining a fungal biomass. Furthermore, the fungal biomass obtained by the method of the present disclosure is edible.

The term “growth carbon source” as used herein refers to a carbon source, which is used while culturing genetically changed genomes of filamentous fungus on a medium. The term “fermentation carbon source” as used herein refers to a carbon source, which is used during fermenting the pre-cultivated improved fungal strains and the fermentation carbon source being comprised in a feedstock. The growth carbon source and the fermentation carbon sources may have the same or similar composition. Alternatively, the growth carbon source and the fermentation carbon sources may have different composition. Both, the growth carbon source and the fermentation carbon source may comprise a mixture of different carbon sources, such as residual sugars, oligosaccharides (maltose) and glycerol. Furthermore, the growth carbon source and the fermentation carbon source may derive from side streams from bioethanol production, vinasse or stillage (residues from ethanol distillation).

The term “fungal biomass” as used herein refers to a total quantity or weight (preferably, a dry cell weight) of fungi (such as a filamentous fungi) in a given volume thereof. Moreover, the feedstock may include a mixture of fermentation carbon sources (such as glucose, oligosaccharides, glycerol, and the like), a vinasse, a thin stillage, or a total dissolved carbon sources content.

In an embodiment, the filamentous fungus is selected to be at least from selected from Trichocomaceae, Nectriaceae, one fungus family Mucoraceae. In this regard, the filamentous fungus belongs to division: Ascomycota. Moreover, the filamentous fungus belongs to genera of the family Trichocomaceae such as Aspergillus, Paecilomyces, and Penicillium, genera of the family Nectriaceae such as Fusarium, and genera of the family Mucoraceae such as Rhizopus. Notably, the Trichocomaceae are a family of fungi in the order Eurotiales. Taxa are saprobes with aggressive colonization strategies, adaptable to extreme environmental conditions. Furthermore, the Nectriaceae comprise a family of fungi in the order Hypocreales. Furthermore, the Mucoraceae are a family of fungi of the order Mucorales, characterized by having the thallus not segmented or ramified. Pathogenic genera include Absidia, Apophysomyces, Mucor, Rhizomucor, and Rhizopus. Using the filamentous fungus selected from the abovementioned families have shown improved capacity to use a mixture of fermentation carbon sources supplied via low-value side streams during fermentation procedure.

In an embodiment, the filamentous fungus is selected to be Paecilomyces variotii, more preferably Paecilomyces variotii strain KCL-24 (Pekilo). The Paecilomyces variotii can utilize a wide spectrum of organic substances for growth thereof and energy generation, with an improved feedstock utilization profile during batch fermentation and fast dilution rates as in continuous fermentation thereof. Moreover, the Paecilomyces variotii is an asexual state of Byssochlamys spectabilis, that is a member of the Phylum Ascomycota (Family Trichocomaceae). Furthermore, the Paecilomyces variotii is a fast growing, producing powdery to suede-like colonies that are yellow-brown or sand-colored. Additionally, the Paecilomyces variotii strain KCL-24 may be used in an aerobic fungal fermentation for mycoprotein production using growth medium containing mixture of different growth carbon sources including residual sugars, oligosaccharides such as maltose and glycerol.

Notably, the fungal biomass may be essential to various domestic and industrial processes. Herein, the fungal biomass is essential for its protein content, referred to as mycoprotein.

In an embodiment, the fungal biomass of the present disclosure may be used as a food ingredient or in whole foods. It will be appreciated that the fungal biomass may be used as an alternative source of protein for human nutrition or animal feed. Moreover, the fungal biomass may be a valuable source of bioactive compounds such as antioxidants, minerals, polyunsaturated fatty acids and fibers (such as β-glucans) which enhance health benefits and reduce the risk of developing certain diseases. Optionally, the edible fungal biomass may find application in preparation of various food ingredients or whole foods including, but not limited to, bakery products, beverages, nutraceuticals, and so forth. The fungal biomass obtained by the aforementioned method is edible and may find application as a mycoprotein with high protein content as compared to the conventional fungal biomass obtained by conventional methods of producing the same.

The method comprises culturing genetically changed genomes of filamentous fungus on a medium, which comprises non-metabolizable glucose analogue and a growth carbon sources comprising glycerol. In this regard, the genetically changed genomes of filamentous fungus are selected based on a difference in at least one of: a genotype, a phenotypic trait.

In an embodiment, the isolated improved fungal strains have a disrupted carbon catabolite repression resulting in improved feedstock utilization profile thereof. Moreover, the improved feedstock utilization profile results in low-cost production of fungal biomass, using aerobic fungal fermentation, with a high protein content therein. Moreover, the filamentous fungus may be suitable for a single cell protein, namely mycoprotein, use. The compound inducing carbon catabolite repression may be selected from free amino acids. Furthermore, the non-metabolizable compound inducing glucose repression may be a non-metabolizable glucose analogue.

The term “carbon catabolite repression” as used herein refers to a set of metabolic processes that involves break down of molecules into smaller units. Moreover, the carbon catabolite repression allows the filamentous fungus to adapt quickly to any preferred carbon and energy source first. In this case, the filamentous fungus may inhibit synthesis of enzymes and transporters involved in catabolism of carbon and energy sources other than the preferred carbon and energy source. However, disruption of such mechanism enables the filamentous fungus to adapt to non-preferred carbon and energy sources without any bias. It will be appreciated that disrupted carbon catabolic repression may increase the glycerol or maltose utilization capacity of the improved filamentous fungus strains.

In an embodiment, the method further comprises mutagenesis of filamentous fungus for genetically changing its genomes before selecting genetically changed genomes. In this regard, the filamentous fungus P. variotii strain KCL-24 is subjected to mutagenesis. The term “mutagenesis” as used herein, relates to a technique of inducing mutations in an organism via a spontaneous natural process or by artificially exposing the organism to mutagens using laboratory procedures. Typically, a parental (or wild-type) P. variotii strain KCL-24 is subjected to mutagenesis in order to produce a mutated P. variotii strain KCL-24 exhibiting a different phenotype, such as disrupted carbon catabolite repression, from that is exhibited by the parental (or wild-type) P. variotii strain KCL-24. Typically, the mutagenesis may be performed by exposing the parental (or wild-type) P. variotii strain KCL-24 to a non-lethal quantity (that does not kill 100% of the strain population) of a mutagenic chemical or a physical mutagen. Optionally the mutagenic chemical may be selected from N-methyl-N-nitrosourea (NMU), methyl methane sulfonate (MMS), N-methyl-N′-nitro-N-nitrosoguanidine (MNNG) and ethyl methanesulphonate (EMS) nitrous acid (NA), diepoxybutane (DEB), 1, 2, 7, 8-diepoxyoctane (DEO), 4-nitroquinoline 1-oxide (4-NQO), 2-methyloxy-6-chloro-9(3-[ethyl2-chloroethyl]-aminopropylamino)-acridinedihydrochloride (ICR-170), 2-amino purine (2AP), and hydroxylamine (HA). Preferably, the mutagenic chemical is ethyl methanesulphonate (EMS). The physical mutagen may be selected from at least one of: ultraviolet (UV) radiation, gamma rays or X-rays.

In an embodiment, genetically changed genomes of filamentous fungus are obtained via mutagenesis by UV-radiation. In this regard, the filamentous fungus may be exposed to UV-radiation for introduction of random genetic changes in the genome. Moreover, the UV-radiation exposed mutants are subsequently plated on a solid agar plate containing a mix of the non-metabolizable glucose analogue and the growth carbon source comprising glycerol. Optionally, the resulting mutants with complete or partial loss of glucose repression mechanism may start to utilize the non-preferred carbon sources in the said conditions leading to a colony appearance. Optionally, on the other hand, the cells with functional glucose repression mechanism may restrict growing thereof. Optionally, the obtained clones may be subjected to a series of tests for selection of the most promising clones. Optionally, the clones showing fast growth on varying non-preferred carbon/energy sources were selected for future use. According to the test results, the genetically changed genomes of filamentous fungus obtained via mutagenesis by UV-radiation were most promising source to culture improved fungal strains, which would be able to utilize high concentration of glycerol during fermentation.

In an embodiment, the non-metabolizable glucose analogue is selected to be 2-deoxy-glucose (2-DG). Typically, 2-DG is a chemical mimicking glucose but that cannot be metabolized by fungal cells. It will be appreciated that the 2-deoxy-glucose is a glucose molecule having the 2-hydroxyl group replaced by hydrogen, in order to prevent further glycolysis. Moreover, the 2-Deoxy-glucose acts to competitively inhibit the production of glucose-6-phosphate from glucose at the phosphoglucoisomerase level. In this regard, the selection of genetically changed genomes of filamentous fungus for growth on medium comprising the non-metabolizable glucose analogue and growth carbon source comprising glycerol results in induction of a carbon catabolite repression and thereby selection of genetically changed genomes of filamentous fungus having disrupted carbon catabolite repression.

In an embodiment, at least one of the growth carbon source and the fermentation carbon source is selected to be at least one of xylo-, malto-1 cello-, and raffinose-derived oligosaccharides. Typically, oligosaccharides comprise from 2 up to 10 saccharide units. The oligosaccharides are obtained from biowastes, such as rice straw, husk, spent coffee grounds, sugarcane bagasse, spent tea leaves, fruits and vegetables peel, corn stalk, corn stover, de-oiled meals, brewer's spent grains, and so forth. The xylo-oligosaccharides (XOS) comprise a C5 (where C is a quantity of carbon atoms in each monomer) structure. The malto-oligosaccharides comprise a (1-4) linked D-glucose. The cello-oligosaccharides are water soluble oligosaccharides that are readily converted to glucose by β-glucosidase. The oligosaccharides comprise for example maltose and lactose. The raffinose-derived oligosaccharides (RFOS) comprise D-galactose.

In an embodiment, at least one of the growth carbon source and the fermentation carbon source further comprises at least one of selected from glucose, galactose, xylose, acetic acid, lactic acid, formic acid, furfural. It will be appreciated that by nature, microbial organisms, including those cultivated for SCP production, preferably first use most rapidly metabolizable carbon sources i.e. free sugars (namely, preferred carbon sources) and only then begin to utilize other carbon sources such as glycerol and oligosaccharides. In this regard, the glucose is a preferred carbon source for the fungal biomass growth than the galactose or the xylose. Optionally, the said carbon sources may be used in an aerobic fungal fermentation for mycoprotein production.

Optionally, mutagenesis may be confirmed using for example RNA sequencing analysis between the parental (or wild-type) and improved (namely mutated) P. variotii strain KCL-24. Expression levels of some most essential glycerol catabolism genes for the parental (or wild-type) and improved (namely mutated) P. variotii strain KCL-24 confirm the increase for improved strains. The embodiment where mutagenesis is carried out with UV-radiation and where a growth carbon source comprising glycerol is used, provides the best improved strains.

Moreover, the method comprises isolating improved fungal strains from the cultured genetically changed genomes, wherein the improved fungal strains exhibit growth on the medium. In this regard, the isolation of improved fungal strains of genetically changed genomes of filamentous fungus may be performed sequentially and iteratively on a solid (or a liquid) medium. The purpose of this step is to eliminate the fungal strains, which did not exhibit any growth from the improved fungal strains, as the non-growing fungal strains would not be able to utilize glycerol comprising carbon sources.

In an embodiment, the isolation of improved fungal strains of genetically changed genomes of filamentous fungus may be performed sequentially and iteratively on a solid (or a liquid) medium to obtain pure colonies as well as to assess the stability of the desired genotype or phenotype, such as disrupted carbon catabolite repression, under conditions more approximating a commercial cultivation scheme. Optionally, the pure colonies may further be inoculated using a liquid media, such as TAP (Tris-Acetate-Phosphate), High Salt Medium™ (HSM™) plus glucose (for example, having 1% w/v glucose). It will be appreciated that said isolated improved fungal strains of genetically changed genomes of filamentous fungus show growth improvement on feedstocks containing a mixture of different carbon sources.

Optionally, said isolated improved fungal strains of genetically changed genomes of filamentous fungus may be genetically stable. The term “genetically stable” as used herein, refers to a characteristic of a species or a strain/isolate to resist changes and maintain its genotype over multiple generations or cell divisions, ideally hundreds to thousands. In an example, the genetically stable strains of P. variotii strain KCL-24 are genetically determined to disrupt carbon catabolite repression and show growth improvement on feedstocks containing a mixture of different carbon sources, comprising galactose, xylose, maltose, and so on. Furthermore, the genetically stable strain of P. variotii strain KCL-24 does not revert to characteristics associated with the parental (or wild-type) P. variotii strain KCL-24 even under alternate growth conditions and over time (such as, over multiple hundreds of generations of cultivation).

Optionally, the fermentation carbon source comprising glycerol is derived from at least one of selected from thin stillage, vinasse. The thin stillage or the vinasse comprises of a mixture of different carbon sources such as residual sugars, oligosaccharides (maltose) and glycerol. The fermentation carbon source comprising glycerol selected from above examples are widely used low value dilute side-streams from which fungal biomass can be economically produced by combining these with the genetically changed genomes of filamentous fungus. In an embodiment, the content of glycerol in the fermentation carbon source is 25-75% of total weight of the fermentation carbon source, thus the content of glycerol in the fermentation carbon source may be from 25, 30, 35, 40, 45, 50, 55, 60, 65 or 70% of total weight up to 30, 35, 40, 45, 50, 55, 60, 65, 70 or 75% of total weight of the fermentation carbon source. Typically, the fermentation carbon source is above 40%. High content of glycerol is possible due to the use of improved fungal strains, which are able to utilize glycerol. The improved fungal strains of the present disclosure enable utilization of glycerol during fermentation, when fermentation carbon sources comprising glycerol are present. The resulting fungal biomass is edible due to high utilization of glycerol content.

In an embodiment, the content of glycerol in the fermentation carbon source is 40-75% of total weight of the fermentation carbon source. Thus, the content of glycerol in the fermentation carbon source may be from 40, 45, 50, 55, 60, 65 or 70% of total weight of the fermentation carbon source up to 30, 35, 40, 45, 50, 55, 60, 65, 70 or 75% of total weight of the fermentation carbon source. Using the fermentation carbon source comprising glycerol more than 40% of total weight of the fermentation carbon source is possible only due to the improved fungal strains, as the improved fundal strains have shown the ability to utilize glycerol from fermentation carbon sources obtained for example from thin stillage or vinasse. The mentioned fermentation carbon sources are normally not optimal for microbial growth, which results in lower productivities and yields than could be achieved. However, according to the test results the improved fungal strains obtained by culturing with non-metabolizable glucose analogue and a growth carbon source comprising glycerol are able to utilize such fermentation carbon sources, in which the glycerol content is above 40% of total weight of the fermentation carbon source. Therefore, a fungal biomass obtained by the method is also edible.

Furthermore, the method comprises pre-cultivating the isolated improved fungal strains. Herein, pre-cultivation may be aerobic cultivation. The pre-cultivation is for generating enough biomass to inoculate the batch or continuous fermentation. It will be appreciated that the pre-cultivation of the isolated improved fungal strains may also reduce the total process time required for the fermentation thereof.

Furthermore, the method comprises fermenting the pre-cultivated fungal strains with a feedstock to obtain a fermentation broth, wherein the feedstock comprises a fermentation carbon source comprising glycerol. Herein, the term “fermenting” as used herein refers to a process of growing or cultivating the pre-cultivated fungal strains in a suitable vessel under optimal growth conditions. Optionally, the optimal growth conditions may comprise an optimal temperature ranging from 25 to 40° C., e.g. 37° C., an agitation ranging from 1000 to 1500 rpm, e.g. 1200 rpm, aeration at a flow rate in a range of 0.1 to 1 VVM (volume per volume minute), e.g. 0.3 VVM, pH 4.6, different dilution rates ranging from low to high dilution rates, e.g. rates >0.3 h⁻¹, in continuous fermentation. Optionally, the fermentation may be aerobic fermentation. Optionally, the fermentation may be anaerobic fermentation.

The term “fermentation broth” as used herein refers to a complex mixture of various components present within the bioreactor as a desired product. In this regard, the pre-cultivated isolated fungal strains are inoculated with a given amount of the feedstock in the bioreactor.

In an embodiment, fermenting is carried out in a bioreactor. The term “bioreactor” as used herein refers to a vessel intended for biological and/or biochemical reactions required for culturing cells, growing micro-organisms, production of biomolecules of pharmaceutical interest and production of the fermentation broth, under defined and controlled physical and chemical conditions. The bioreactor may have a shape such as cylindrical, conical, cuboidal or cubical. Optionally, the shape of the bioreactor may be selected based on a desired volume and usage of the bioreactor. Optionally, the volume of the bioreactor is for example 2 litres, 5 litres, 10 litres, 100 litres, 200 litres, 1000 litres, 5000 litres, 10000 litres, 20000 litres, 50000 litres, 100000 litres, 200000 litres and the like.

Optionally, the bioreactor is fabricated from a material that is inert to contents to be processed in the bioreactor. In an example, the material used for fabrication may be stainless steel, other suitable metals or alloys, glass material, fibres, ceramic, plastic materials and/or combinations thereof. Moreover, the fabrication material is typically waterproof and strong enough to withstand abrasive effects of various biological, biochemical and/or mechanical processes, such as micro-organisms concentrations, biomass productions, agitation forces, aeration forces, operating pressures, temperatures, acids, alkali and so forth. Furthermore, the bioreactor should preferably be such that it withholds the sterilization conditions, for example steam sterilization with water vapor at 121° C. and a pressure of 2.5 bar. Typically, the bioreactor may have an adequate thickness to hold a weight of the fermentation broth, and carry out various biological, biochemical and/or mechanical processes.

In an embodiment, the bioreactor is selected to be at least one of a stirred tank bioreactor, an airlift bioreactor. The stirred tank bioreactor typically use internal mechanical agitation for mixing various components (having different phase compositions) therein. The airlift bioreactor typically is similar to the stirred tank bioreactor with only difference that the airlift bioreactor employs compressed air for aeration and agitation.

In an embodiment, fermentation is a batch fermentation. In this regard, when in use, the feedstock is inoculated to a fixed volume of medium in the bioreactor for single cell protein production. Moreover, with microbial growth, the nutrients are gradually consumed. The fermentation broth is removed at the end and the fungal biomass may be harvested. Optionally, in batch fermentation, biomass concentrations (such as cellular dry weight (CDW)) may be monitored by determining the dry weight of the fungal biomass collected by a filtration.

Optionally, a fed-batch fermentation may be used that is a modified version of batch fermentation. Optionally, the isolated improved fungal strains are inoculated and grown under batch regime for a certain amount of time, then nutrients are added to the fermenter in increments throughout the remaining duration of fermentation to feed thereof.

In an embodiment, fermentation is a continuous fermentation. The term “continuous fermentation” as used herein refers to a process that involves continuous addition of a fresh volume of feedstock into the bioreactor, while used volume of the feedstock (growth medium and cells) are harvested at the same time. In this regard, the consumed nutrients may be replaced and toxic metabolites may be removed from the culture. Notably, the continuous fermentation begins with a short batch fermentation but without harvesting the biomass. Moreover, during the continuous fermentation, the fungal biomass may be taken from the outflow and collected by filtration.

In an embodiment, the feedstock is fed and the fermentation broth is collected at the same rate in the continuous fermentation. It will be appreciated that, when in use, the addition of the feedstock and removal of the fermentation broth are at the same rate (such as same dilution rate), the culture volume and biomass concentration at steady state stay constant. Optionally, the continuous fermentation may begin with a short batch fermentation without harvesting the fungal biomass. Optionally, the continuous fermentation may be continued for weeks or months.

In an embodiment, the dilution rate is selected to be from 0.3 h⁻¹ up to 0.5 h⁻¹. Optionally, the dilution rate may be selected to be from 0.3, 0.35, 0.4 or 0.45 up to 0.35, 0.4, 0.45 or 0.5 h⁻¹. Optionally, during the continuous fermentation, a high dilution rates that is greater than 0.3 h⁻¹ may be used. Beneficially, the high dilution rate decreases residence time of the fungal biomass in the bioreactor while speeding up the fermentation process.

Furthermore, the method comprises collecting the fermentation broth and separating the fungal biomass from the fermentation broth. In this regard, the fermentation broth is collected as a product from the bioreactor. It will be appreciated that the fermentation broth may have a higher concentration of the fungal biomass. Moreover, the fungal biomass is separated from the fermentation broth to obtain the desired fungal biomass with high concentration of the mycoprotein therein. Moreover, the said fungal biomass may have a higher moisture content starting from 9 percent such that more fermentation carbon sources may be consumed during the fermentation. Furthermore, optionally, the said fungal biomass may be in a solid state having a low moisture content such as 4 to 8 percent.

In an embodiment, the separating is carried out by at least one of selected from filtration, centrifugation. Typically, the filtration process involves a physical separation of solid matter and fluid from a mixture using a filter medium. Optionally, the filtration process may vary depending on whether the fungal biomass is dissolved in the fluid phase or suspended as a solid. Moreover, centrifugation process typically involves the use of the centrifugal force to separate particles from a solution according to their size, shape, density, medium viscosity and rotor speed. Optionally, the method of separation may be selected based on the application thereof.

In an embodiment, the method further comprises drying the fungal biomass. The term “drying” as used herein refers to a mass transfer process consisting of the removal of water or another solvent by evaporation from a solid, semi-solid or liquid. Optionally, the drying process may be used as a final production step before selling or packaging the fungal biomass. In this regard, the fungal biomass is dried in order to be consumed in at least one of: a powder form, a tablet form, any other suitable dry form. Optionally, a dryer may be implemented to dry the fermentation broth obtained from the bioreactor.

In an embodiment, drying is selected to be at least one of selected from hot air drying, freeze drying, contact drying. The hot air drying (HAD) typically involves heat to be transferred from a hot air to the product by convection, and evaporated moisture is transported to the air also by convection. Typically, the freeze drying, also known as lyophilization or cryodesiccation, is a low temperature dehydration process that involves freezing the product, lowering pressure, then removing the ice by sublimation. Typically, the contact drying is implemented using a contact dryer. Optionally, when in use, the fermentation broth may be fed or placed on to a vacuum belt conveyor of the contact dryer. Optionally, the contact dryers may supply the heat via a heated vacuum plate (with transport belt) from the bottom side of the material. It will be appreciated that the contact drying may be an economical and an efficient process of drying.

The present disclosure also relates to the fungal biomass as described above. Various embodiments and variants disclosed above apply mutatis mutandis to the fungal biomass.

### EXAMPLES

### Example Way(s) of Generating the Improved Fungal Strain

The filamentous fungus P. variotii strain KCL-24 was exposed to UV-radiation for introduction of random genetic changes in the genome. The UV-exposed mutants were plated on a solid agar plate containing a mix of varying non-preferred carbon sources such as glycerol in growth carbon sources. The plates were supplemented with 2-deoxy-glucose (2-DG), a chemical mimicking glucose but which cannot be metabolized by fungal cells, for induction of carbon catabolite repression. The resulting mutants with complete or partial loss of glucose repression mechanism start to utilize non-preferred carbon sources in these conditions leading to colony appearance. On the other hand, the cells with functional glucose repression mechanism do not grow. The obtained clones were subjected to series of tests for selection of the most promising clones. The clones showing fast growth on varying non-preferred carbon/energy sources were selected for future use.

In order to confirm metabolic changes, RNA sequencing analysis was carried out and compared between the parental and improved strain using continuous fermentation conditions as described in the next example. Expression levels of the most essential glycerol catabolism genes are summarized below in Table 1.

TABLE 1
Relative gene expression between parental (or wild-type) and
improved (namely mutated) P. variotii strain KCL-24

| Gene | Parental | Improved | |
|---|---|---|---|
| ID | strain | strain | Putative function |
| 295783 | 100% | 118% | Glycerol kinase |
| 407650 | 100% | 121% | Glycerol 3-phosphate DH (FAD) |
| 408591 | 100% | 72% | Glycerol 2-DH (NADP) |
| 466146 | 100% | 111% | Glycerol 2-DH (NADP) |
| 487795 | 100% | 121% | Glycerol 2-DH (NADP) |
| 403873 | 100% | 115% | Glycerol 2-DH (NADP) |
| 108972 | 100% | 147% | Aldehyde dehydrogenase |
| 445807 | 100% | 128% | Aldehyde dehydrogenase |
| 420091 | 100% | 145% | Aldehyde dehydrogenase |
| 482952 | 100% | 147% | Aldehyde dehydrogenase |
| 481808 | 100% | 125% | Glycerate 3-kinase |
| 403876 | 100% | 70% | Dihydroxyacetone kinase |
| 498854 | 100% | 345% | Dihydroxyacetone kinase |
| 63621 | 100% | 270% | Glycerol permease (transporter) |
| 109785 | 100% | 82% | Glycerol permease (transporter) |
| 185636 | 100% | 87% | Glycerol permease (transporter) |
| 438872 | 100% | 3238% | Glycerol permease (transporter) |
| 440074 | 100% | 116% | Glycerol permease (transporter) |

### Example Use(es) of the Improved Fungal Strain to Demonstrate the Benefit

The improved P. variotii strain was used in batch and the following continuous fermentation for the production of mycoprotein. A stirred tank bioreactor having a working volume of 3 liters may be used for producing the fungal biomass in presence of the feedstock comprising a mixture of ammonium sulfate (8 grams/liter (g/l)), Magnesium sulfate heptahydrate (MgSO4*7H2O (0.15 g/l)), sodium chloride (NaCl (1 g/l)), calcium chloride (CaCl₂) (0.45 g/l)), monopotassium phosphate (KH₂PO₄ (0.45 g/l)), Vogel's trace elements (0.1 g/l), yeast extracts (2.15 g/l), molasses (5 g/l), glycerol (8 g/l), maltose (2 g/l), and lactic acid (3 g/l).

The fermentation parameters were as follows: 37° C., agitation 1200 rpm, aeration 0.9 l/min, pH 4.6, different dilution rates in the continuous fermentation. In batch fermentation, biomass concentrations (cellular dry weight=CDW) were monitored by determining the dry weight of the samples collected by filtration.

A Table 2 below provides the concentrations of fungal biomass and fermentation carbon sources in an exemplary batch fermentation at a given time point in batch fermentation. In an example, at a given point of time such as 16.7 hour, the batch fermenter or the batch bioreactor may comprise a 1.04 g/l cellular dry weight concentration of the fungal biomass, 7.63 g/l of glycerol and 0.95 g/l of maltose, and 0.84 g/l of glucose. In another embodiment, the improved strain at a given point of time such as 16.33 hours, the batch bioreactor may comprise 2.69 g/l cellular dry weight concentration of the fungal biomass, 6.30 g/l of glycerol and 0.00 g/l of maltose, and 0.52 g/l of glucose. According to the table 2, that with the improved strain, concentrations of glycerol, maltose and glucose are decreasing more rapidly during fermentation.

TABLE 2
Biomass concentration and fermentation carbon
sources in an exemplary batch fermentation

| | | CDW | Glycerol | Maltose | Glucose |
|---|---|---|---|---|---|
| Strain | Time (h) | (g/l) | (g/l) | (g/l) | (g/l) |
| parental | 0 | 0.00 | 7.60 | 1.59 | 1.10 |
| | 16.7 | 1.04 | 7.63 | 0.95 | 0.84 |
| | 18.45 | 2.35 | 7.46 | 0.00 | 0.78 |
| | 20.63 | 3.76 | 7.30 | 0.00 | 0.00 |
| | 22.1 | 4.79 | 5.05 | 0.00 | 0.00 |
| improved | 0 | 0.00 | 7.66 | 1.63 | 1.11 |
| | 16.33 | 2.69 | 6.30 | 0.00 | 1.90 |
| | 18.17 | 4.77 | 3.94 | 0.00 | 0.52 |
| | 20.37 | 7.84 | 0.00 | 0.00 | 0.00 |
| | 21.97 | 7.24 | 0.00 | 0.00 | 0.00 |

A Table 3 below provides different dilution rates, the fungal biomass concentration, fermentation carbon sources and the biomass crude protein content in an exemplary continuous fermentation. Crude protein content was determined using Dumas method. Concentrations of different carbon sources in fermentation carbon sources were analyzed from the samples using a High-Performance Liquid Chromatography (HPLC). According to the table 3, using the improved strain, the glycerol concentration was low even at low dilution rates during continuous fermentation.

TABLE 3
Dilution rate, biomass concentration, fermentation
carbon sources and the biomass crude protein content
in an exemplary continuous fermentation

| | Dilution | CDW | Glycerol | Crude protein |
|---|---|---|---|---|
| Strain | rate | (g/l) | (g/l) | % |
| parental | 0.20 | 6.23 | 4.85 | 57.21 |
| | 0.20 | 6.625 | 3.4 | 56.2 |
| | 0.29 | 4.54 | 6.15 | 61.6 |
| | 0.29 | 4.1 | 5.99 | 60.92 |
| | 0.29 | 4.715 | 5.83 | 57.14 |
| | 0.29 | 4.6 | 5.79 | 57.45 |
| | 0.30 | 4.465 | 6.08 | 55.93 |
| improved | 0.18 | 8.67 | 0 | 58.97 |
| | 0.18 | 7.95 | 0 | 57.45 |
| | 0.27 | 7.02 | 0 | 63.4 |
| | 0.27 | 6.79 | 0 | 61.07 |
| | 0.31 | 7.925 | 0 | 61.67 |
| | 0.33 | 6.595 | 1.21 | 63.87 |

### DETAILED DESCRIPTION OF THE DRAWINGS

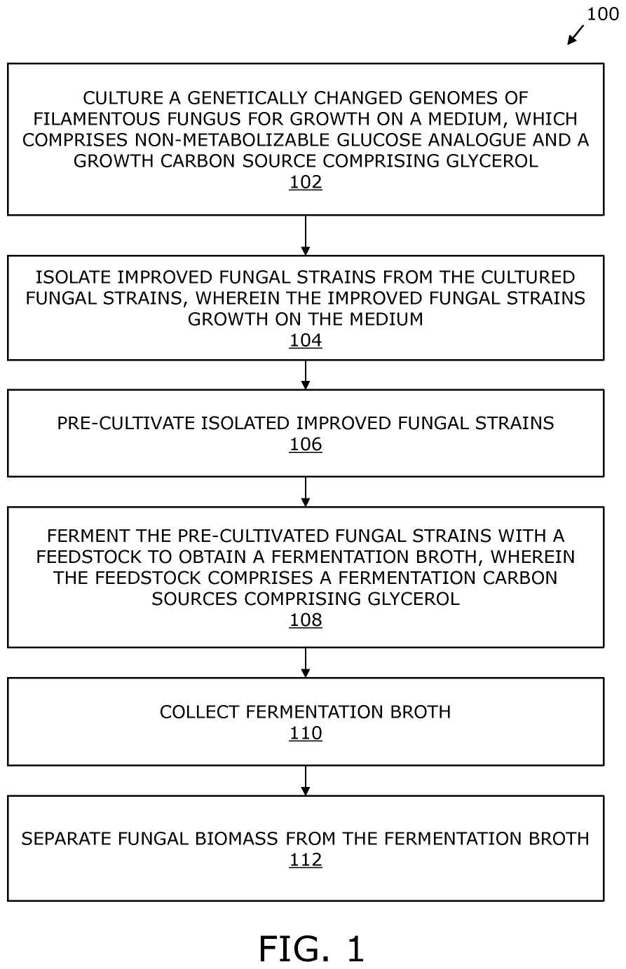

Referring to FIG. 1, illustrated is a flowchart 100 of steps of a method of producing a fungal biomass, in accordance with an embodiment of the present disclosure. At step 102, a genetically changed genomes of filamentous fungus are cultured on a medium, which comprises non-metabolizable glucose analogue and a growth carbon source comprising glycerol. At step 104, improved fungal strains from the genomes of filamentous fungus, wherein the improved fungal strains exhibit growth on the medium, are isolated. At step 106, the isolated improved fungal strains are pre-cultivated. At step 108, the pre-cultivated fungal strains are fermented with a feedstock to obtain a fermentation broth, wherein the feedstock comprises a fermentation carbon source comprising glycerol. At step 110, the fermentation broth is collected. At step 112, the fungal biomass is separated from the fermentation broth.

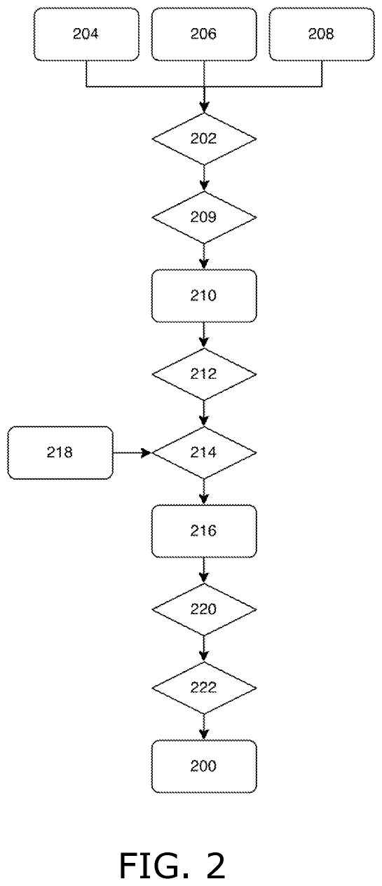

Referring to FIG. 2, shown is a schematic illustration of steps of a method of producing a fungal biomass 200, in accordance with an embodiment of the present disclosure. Rounded rectangles represent the various objects in the production process and rhombuses represent the process steps. In culturing step 202, the genetically changed genomes of filamentous fungus 204 are cultured on a medium, which comprises non-metabolizable glucose analogue 206 and a growth carbon source 208 comprising glycerol. At the isolating step 209, the improved fungal strains 210 from the cultured genetically changed genomes, wherein the improved fungal strains exhibit growth on the medium, are isolated. At pre-cultivating step 212 the isolated improved fungal strains are pre-cultivated. At the fermenting step 214, the pre-cultivated improved fungal strains are fermented with a feedstock to obtain a fermentation broth 216, wherein the feedstock comprises a fermentation carbon source 218 comprising glycerol. At the collecting step 220, the fermentation broth is collected. At the separating step 222, the fungal biomass is separated from the fermentation broth.

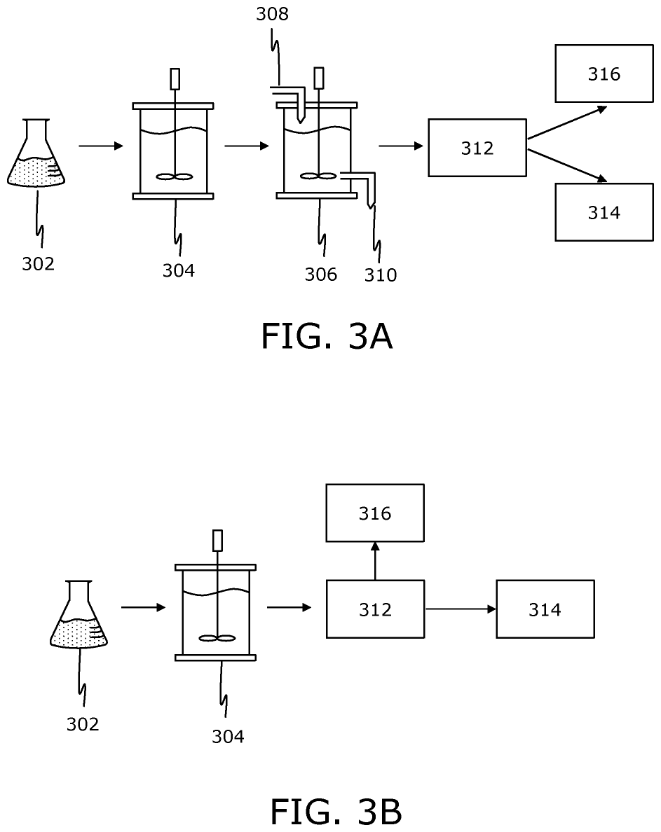

Referring to figures FIG. 3A and FIG. 3B, illustrated are two embodiments for steps of a method of cultivating the isolated fungal strains with a disrupted carbon catabolite repression for producing a fungal biomass. As shown in figures FIG. 3A and FIG. 3B, a flask 302 is used for pre-cultivating the isolated improved fungal strains. Moreover, as shown in FIG. 3A, a combination of a batch fermentation implemented via a batch bioreactor 304 and a continuous fermentation implemented via a continuous bioreactor 306 is employed for fermenting the pre-cultivated fungal strains therein to obtain a fermentation broth. Furthermore, the product obtained from the batch bioreactor 304 may be used as a feedstock for the continuous bioreactor 306. In this regard, the feedstock may be provided through an inlet 308 of the continuous bioreactor 306 and the fermentation broth may be collected from an outlet 310 of the continuous bioreactor 306. Furthermore, the continuous reactor 306 may provide the fermentation broth with a higher biomass concentration. Moreover, FIG. 3B shows another embodiment of the method for producing the fungal biomass, wherein only a batch fermentation implemented via the batch bioreactor 304 is used for producing the fermentation broth.

Additionally, the fungal biomass is separated from the fermentation broth by filtration using a mechanical filtering unit 312. Furthermore, the mechanical filtering unit 312 may provide the fungal biomass in a solid dry state 314 or a liquid state 316.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of producing a fungal biomass comprising:
culturing genetically changed genomes of filamentous fungus on a medium, which comprises non-metabolizable glucose analogue and a growth carbon source comprising glycerol,
isolating improved fungal strains from the cultured genetically changed genomes, wherein the improved fungal strains exhibit growth on the medium,
pre-cultivating the isolated improved fungal strains,
fermenting the pre-cultivated improved fungal strains with a feedstock to obtain a fermentation broth, wherein the feedstock comprises a fermentation carbon source comprising glycerol,
collecting the fermentation broth, and
separating the fungal biomass from the fermentation broth.

2. The method according to claim 1, wherein the improved fungal strains have disrupted carbon catabolite repression.

3. The method according to claim 1, wherein the genetically changed genomes of filamentous fungus are obtained via mutagenesis by UV-radiation.

4. The method according to claim 1, wherein the content of glycerol in the fermentation carbon source is 25-75% of total weight of the fermentation carbon source.

5. The method according to claim 1, wherein the content of glycerol in the fermentation carbon source is 40-75% of total weight of the fermentation carbon source.

6. The method according to claim 1, wherein at least one of the growth carbon source and the fermentation carbon source is selected from at least one of xylo-, malto-, cello-, and raffinose-derived oligosaccharides.

7. The method according to claim 1, wherein at least one of the growth carbon source and the fermentation carbon source further comprises at least one of selected from glucose, galactose, xylose, acetic acid, lactic acid, formic acid, furfural.

8. The method according to claim 1, wherein the non-metabolizable glucose analogue is 2-deoxy-glucose.

9. The method according to claim 1, wherein the filamentous fungus is selected to be at least from one fungus family selected from Trichocomaceae, Nectriaceae, Mucoraceae.

10. The method according to claim 1, wherein fermentation is a batch fermentation.

11. The method according to claim 1, wherein fermentation is a continuous fermentation.

12. The method according to claim 11, wherein the dilution rate is from 0.3 h⁻¹ up to 0.5 h⁻¹.

13. The method according to claim 1, wherein the fermentation carbon source is derived from at least one of selected from thin stillage, vinasse.

14. The method according to claim 1, wherein the separating is carried out by at least one of selected from filtration, centrifugation.

15. The method according to claim 1, wherein the method further comprises drying the fungal biomass.

16. The method according to claim 15, wherein drying is selected to be at least one of selected from hot air drying, freeze drying, contact drying.

17. The method according to claim 1, wherein the fermenting is carried out in a bioreactor selected from at least one of a stirred tank bioreactor, an airlift bioreactor.

18. The method according to claim 1, wherein the filamentous fungus is selected to be Paecilomyces variotii, more preferably Paecilomyces variotii strain KCL-24.

19. Use of the fungal biomass according to claim 1 as a food ingredient or in whole foods.
